(19) United States
(12) Patent Application Publication
(10) Pub. No.: US 2021/0238118 A1
(43) Pub. Date: Aug. 5, 2021

(54) METHOD FOR PRODUCING ACETYLACETONATE FROM A HYDRATED OR ANHYDROUS CHEMICAL ELEMENT

(71) Applicant: COMPAGNIE GENERALE DES ETABLISSEMENTS MICHELIN, Clermont-Ferrand (FR)

(72) Inventor: PHILIPPE LAUBRY, Clermont-Ferrand (FR)

(21) Appl. No.: 16/972,334

(22) PCT Filed: Jun. 5, 2019
(86) PCT No.: PCT/FR2019/051345

§ 371 Date: Dec. 4, 2020

(30) Foreign Application Priority Data
Jun. 5, 2018 (FR) 1854881

(51) Int. Cl. C07C 45/77

(52) U.S. Cl. CPC C07C 45/77

(57) ABSTRACT

A process for the preparation of the hydrated and/or anhydrous acetylacetonate of a chemical element Me, where the chemical element Me is chosen from alkaline earth metals, transition metals and lanthanides, comprises a stage of reaction in an aqueous medium of the Me oxide or hydroxide introduced in the solid form and of acetylacetone, the acetylacetone being in excess with respect to the Me oxide or hydroxide.

The invention relates to a process for the preparation of the hydrated or anhydrous acetylacetonate of a chemical element starting from oxide or hydroxide of the chemical element, from acetylacetone and from water.

The invention also relates to a continuous batchwise process for the synthesis of such a hydrated or anhydrous acetylacetonate of a chemical element.

A known route for the synthesis of cobalt(II) acetylacetonate dihydrate (CoAA₂.2H₂O) is to react acetylacetone in the form of enolate of alkali metal (Na, K) or pseudoalkali metal (ammonium) type with cobalt dichloride. The disadvantage of this reaction is that it releases undesirable compounds, for example sodium chloride.

A process for the preparation of cobalt(II) acetylacetonate dihydrate (CoAA₂.2H₂O) is known from the document U.S. Pat. No. 7,282,573. The cobalt(II) hydroxide used is prepared in aqueous solution by reaction of potassium hydroxide KOH with cobalt(II) acetate tetrahydrate. After filtration and washing with cold water, the Co(OH)₂ is directly used in a bulk reaction (without solvent) with acetylacetone in enol form in slight excess (10% molar) for 30 minutes (exothermic reaction). After cooling in an ice/water bath, the CoAA₂. 2H₂O derivative obtained is filtered off and then dried under vacuum.

This document also describes the preparation of magnesium acetylacetonate dihydrate (MgAA₂.2H₂O) from magnesium chloride in an aqueous medium. In a first step, magnesium hydroxide is formed by reaction between magnesium dichloride and potassium hydroxide. Then, in a second step, the magnesium hydroxide reacts in bulk (without solvent) with acetylacetone in enol form in slight excess to result in magnesium acetylacetonate dihydrate (MgAA₂.2H₂O).

A process for the preparation of cobalt(II) acetylacetonate dihydrate (CoAA₂.2H₂O) is also known from the document CN 1746180. In an aqueous medium, sodium hydroxide NaOH is reacted with CoCl₂ or Co(NO₃)₂ to generate the hydroxide Co(OH)₂, then, without isolating the cobalt hydroxide formed, acetylacetone is added in molar excess with respect to the cobalt (ratio 6 and 8 in the examples). After refluxing for 3 hours at approximately 130° C., followed by cooling, the precipitated cobalt(II) acetylacetonate dihydrate is filtered off, washed with water and dried at 40° C. under vacuum.

However, washing with water is problematic because, as cobalt(II) acetylacetonate dihydrate is soluble in water at a level of 5 g/1, product is lost, all the more so as it is desired to obtain a low content of salts (chlorides, nitrates, sulfates, and the like).

There thus exists a need to have available a process for the synthesis of hydrated or anhydrous acetylacetonate of a chemical element which does not generate undesirable compounds and thus which does not require a washing stage, and which exhibits an improved overall yield.

The documents U.S. Pat. Nos. 6,376,719 and 6,093,844 disclose processes for obtaining anhydrous alkaline earth metal acetylacetonates by the bulk route and drying at high temperature or under very high vacuum. They do not make it possible to obtain a hydrated product.

The Applicant Company has discovered that the objectives which precede could be achieved by means of a process for the preparation of the hydrated or anhydrous acetylacetonate of a chemical element Me, where the chemical element Me is chosen from alkaline earth metals, transition metals and lanthanides, comprising a stage of reaction in an aqueous medium of the Me oxide or hydroxide introduced in the solid form and of acetylacetone, the acetylacetone being in excess with respect to the Me oxide or hydroxide.

The Applicant Company has also discovered that the objectives which precede could be achieved by means of a continuous batchwise process for the synthesis of the hydrated or anhydrous acetylacetonate of a chemical element Me, where the chemical element Me is chosen from alkaline earth metals, transition metals and lanthanides, comprising n successive stages of reaction i in an aqueous medium of the Me oxide or hydroxide introduced in the solid form and of acetylacetone, the acetylacetone being in excess with respect to the Me oxide or hydroxide, filtration being carried out on conclusion of each reaction i, i varying from 1 to n, the liquid filtrate recovered on conclusion of the reaction i′, i′ varying from 1 to n−1, and comprising water, the excess acetylacetone and a fraction of dissolved hydrated Me acetylacetonate, being recycled by addition to the reaction medium of the reaction i′+1, n being greater than or equal to 2.

A subject-matter of the invention is thus a process for the preparation of the hydrated or anhydrous acetylacetonate of a chemical element Me, where the chemical element Me is chosen from alkaline earth metals, transition metals and lanthanides, comprising a stage of reaction in an aqueous medium of the Me oxide or hydroxide introduced in the solid form and of acetylacetone, the acetylacetone being in excess with respect to the Me oxide or hydroxide.

Hydrated Me acetylacetonate is understood to mean a product which comprises one or more water molecules generally denoted by water of crystallization.

Anhydrous Me acetylacetonate is understood to mean a product which does not comprise water molecules.

As explained below, depending on the presence or absence of a drying stage and on the drying conditions, a hydrated, dried hydrated or anhydrous Me acetylacetonate is obtained.

Me oxide or hydroxide is understood to mean the oxidized or hydroxidized chemical element Me.

Me oxide or hydroxide introduced in the solid form is understood to mean, within the meaning of the present invention, that the Me oxide or hydroxide is not introduced in solution.

Mention may be made, among the alkaline earth metals which can be used according to the invention, of magnesium, calcium, strontium and barium.

Mention may be made, among the transition metals which can be used according to the invention, of cobalt, nickel, copper and zinc.

Mention may be made, among the lanthanides which can be used according to the invention, of lanthanum, cerium, praseodymium and neodymium.

The chemical element Me is preferably chosen from cobalt, magnesium, nickel, calcium, neodymium and zinc.

According to a first embodiment, the chemical element Me is cobalt. The compound obtained according to the process according to the invention is then cobalt acetylacetonate dihydrate (CoAA₂.2H₂O). The precipitate of this compound exhibits a salmon pink colour. According to the process according to the invention, this compound is obtained by reaction between cobalt(II) hydroxide (Co(OH)₂) and acetylacetone in water.

According to a second embodiment, the chemical element Me is magnesium. The compound obtained according to the process according to the invention is then magnesium acetylacetonate dihydrate (MgAA₂.2H₂O). The precipitate of this compound exhibits a white colour. According to the process according to the invention, this compound is obtained by reaction between magnesium(II) hydroxide (Mg(OH)₂) or magnesium oxide MgO and acetylacetone in water.

When magnesium oxide is used as starting material, it reacts with water to form magnesium(II) hydroxide (Mg(OH)₂) which in turn will react with acetylacetone in water to form the final product (MgAA₂.2H₂O).

As explained above, the acetylacetone is in excess with respect to the Me oxide or hydroxide.

Preferably, the acetylacetone/Me oxide or hydroxide molar ratio is greater than 2 when the Me oxide or hydroxide is divalent and greater than 3 when the Me oxide or hydroxide is trivalent, more preferably greater than or equal to 4 when the Me oxide or hydroxide is divalent and greater than or equal to 6 when the Me oxide or hydroxide is trivalent, more preferably equal to 6 when the Me oxide or hydroxide is divalent and equal to 9 when the Me oxide or hydroxide is trivalent.

Thus, when the chemical element is cobalt, the reaction scheme is as follows:

Co(OH)₂+2 AAH→CoAA₂.2H₂O

where AAH represents acetylacetone in the enol form.

When the chemical element is magnesium, the reaction scheme is as follows:

Mg(OH)₂+2 AAH→MgAA₂.2H₂O, or else

MgO+H₂O+2 AAH→MgAA₂.2H₂O

where AAH represents acetylacetone in the enol form.

The reaction time is generally between 2 h and 6 h, preferably of the order of 4 h.

On conclusion of the reaction, a filtration stage is generally carried out in order to recover a solid phase comprising the hydrated Me acetylacetonate and a liquid filtrate comprising water, the excess acetylacetone and a dissolved hydrated Me acetylacetonate fraction.

In order to make possible efficient filtration and the recycling of the liquid filtrate, the liquid filtrate must be homogeneous, that is to say exhibit only a single phase. The weight of acetylacetone in the aqueous phase of the liquid filtrate should advantageously remain less than or equal to 15% of the weight of the aqueous phase. Beyond that, filtration proves to be more difficult and the recycling must take into account the fact that the filtrate may be two-phase. The aqueous dilution of the initial reaction medium (Me oxide or hydroxide and acetylacetone in water) must be accordingly adjusted.

On conclusion of the filtering stage, the Me acetylacetonate present in the solid phase is in a hydrated form. It is subsequently generally dried.

This drying can be carried out by applying a vacuum and/or by reducing the vapour pressure by a stream of gas, preferably inert gas, and/or by increasing the temperature of the product in order to remove the free water (obtaining the dried hydrated product), and also the water of crystallization, in the case of an anhydrous final product.

A person skilled in the art will know how to adjust the drying temperature depending on the element Me. For example, when the chemical element is cobalt, the drying temperature is generally less than 75° C. When the chemical element is magnesium, the drying temperature is generally less than 65° C.

The filtration and the drying thus carried out are easy, by virtue of the crystallinity of the Me acetylacetonates obtained.

The product obtained, the hydrated or anhydrous Me acetylacetonate, is in the form of a powder of fine non-agglomerated particles.

This recovered product is very pure, with a purity close to 100%, and does not require any washing because there is no reaction by-product (such as sodium chloride).

The liquid filtrate obtained on conclusion of the filtering stage is advantageously entirely recycled for the following synthesis, as well as the vapours obtained on conclusion of the drying. This recycling operation is repeated at each new synthesis so that there is no loss of the chemical element Me or of acetylacetone.

The condensates obtained during the drying stage contain essentially water and a small amount of acetylacetone. In an industrial implementation, they can advantageously be entirely recycled in the following syntheses.

It is thus not necessary to have a stage of treatment of the waste effluents.

Thus, another subject-matter of the invention is a continuous batchwise process for the synthesis of the hydrated or anhydrous acetylacetonate of a chemical element Me, where the chemical element Me is chosen from alkaline earth metals, transition metals and lanthanides, the said process comprising n successive stages of reaction i in an aqueous medium of the Me oxide or hydroxide introduced in the solid form and of acetylacetone, the acetylacetone being in excess with respect to the Me oxide or hydroxide, filtration being carried out on conclusion of each reaction i, i varying from 1 to n, the liquid filtrate recovered on conclusion of the reaction i′, varying from 1 to n−1, and comprising water, the excess acetylacetone and a fraction of dissolved hydrated Me acetylacetonate, being recycled by addition to the reaction medium of the reaction i′+1, n being greater than or equal to 2.

Continuous batchwise process is understood to mean, within the meaning of the present invention, a process comprising a cycle of successive syntheses.

Mention may be made, among the alkaline earth metals which can be used according to the invention, of magnesium, calcium, strontium and barium.

Mention may be made, among the transition metals which can be used according to the invention, of cobalt, nickel, copper and zinc.

Mention may be made, among the lanthanides which can be used according to the invention, of lanthanum, cerium, praseodymium and neodymium.

The chemical element Me is preferably chosen from cobalt, magnesium, nickel, calcium, neodymium or zinc.

Preferably, n varies from 2 to 60, more preferably from 2 to 40, more preferably from 2 to 20.

Each reaction of the continuous batchwise process according to the invention is as described above concerning the batchwise process according to the invention.

In particular, the acetylacetone/Me oxide or hydroxide molar ratio is greater than 2 when the Me oxide or hydroxide is divalent and greater than 3 when the Me oxide or hydroxide is trivalent, preferably greater than or equal to 4 when the Me oxide or hydroxide is divalent and greater than or equal to 6 when the Me oxide or hydroxide is trivalent, more preferably equal to 6 when the Me oxide or hydroxide is divalent and equal to 9 when the Me oxide or hydroxide is trivalent.

Filtration is carried out after each reaction i. The filtration makes it possible to recover the hydrated acetylacetonate of the chemical element Me and a filtrate. For each filtration i′, the filtrate recovered is used in the following reaction, as indicated above.

Preferably, the weight of acetylacetone in the aqueous phase of the liquid filtrate is less than or equal to 15% of the weight of the aqueous phase.

The continuous batchwise process according to the invention thus makes it possible to recycle the filtrate from each reaction for the synthesis of the hydrated Me acetylacetonate in the following reaction.

The yield is thus virtually quantitative. The process does not require a specific treatment of waste effluents.

The hydrated acetylacetonate of the chemical element Me recovered after the filtration carried out after each reaction is preferably dried.

A further subject-matter of the invention is a process for the preparation of the hydrated or anhydrous acetylacetonate of a chemical element Me, where the chemical element Me is chosen from alkaline earth metals, transition metals and lanthanides, starting from Me oxide or hydroxide and acetylacetone, in which the said Me acetylacetonate is obtained as well as a liquid filtrate containing acetylacetone in the aqueous phase and a condensate, the liquid filtrate being capable of being used for a new preparation of acetylacetonate of the element Me as reactant.

Preferably, the said process comprises a first stage of reaction in an aqueous medium of the oxide or hydroxide of the element Me introduced in the solid form and of acetylacetone, the acetylacetone being in excess with respect to the Me oxide or hydroxide, and a second filtration stage.

The weight of acetylacetone in the aqueous phase of the liquid filtrate is advantageously less than or equal to 15% of the weight of the aqueous phase.

The condensate is capable of being used for a new preparation of acetylacetonate of the element Me as reactant.

The invention is illustrated by the following examples.

### EXAMPLES

The characterization tests used in the examples are as follows.

Mg Level by Complexometry in Magnesium Acetylacetonate

The magnesium contained in magnesium acetylacetonate is released into the aqueous phase by dissolving in acetone and then hydrolysed by a hydrochloric acid solution.

The manipulation consists of a quantitative determination of the magnesium ions (Mg²⁺) of the sample by EDTA in the presence of a coloured indicator: Eriochrome Black T (denoted EBT). The end of quantitative determination is detected by a Phototrode set at 660 nm.

At the start of quantitative determination, the EBT complexes with the magnesium ions present, which gives a purplish colour to the solution.

The magnesium ions complex preferentially with the EDTA. At the end of the quantitative determination, all the magnesium ions will have complexed with the EDTA. The coloured indicator (EBT) will thus recover its free form and its initial colour: bluish. This change in colour is monitored by the abovementioned Phototrode.

The total hardness corresponds to the amount of EDTA (denoted H2Y2-) used to reach the colour change.

Co Level by Complexometry in Cobalt Acetylacetonate

The cobalt contained in cobalt acetylacetonate is released into the aqueous phase by dissolving in acetone and then hydrolysed by a hydrochloric acid solution.

The manipulation consists of a quantitative determination of the cobalt ions of the sample by EDTA in the presence of a coloured indicator: Xylenol Orange. The end of quantitative determination is detected by the visual observation of a change in colour.

At the start of quantitative determination, the Xylenol Orange complexes with the cobalt ions present, which gave the solution a pinkish colour.

The cobalt ions complex preferentially with the EDTA. At the end of the quantitative determination, all the cobalt ions will have complexed with the EDTA. The coloured indicator (Xylenol Orange) will thus recover its free form and its initial colour: orange. This change in colour is monitored visually.

The total hardness corresponds to the amount of EDTA (denoted H2Y2-) used to reach the colour change.

Mg Level by Spectrophotometry in Magnesium Acetylacetonate

Magnesium acetylacetonate is hydrolysed in an acidified aqueous solution (for example with hydrochloric acid) under hot conditions. Once the magnesium acetylacetonate has completely dissolved, the solution thus obtained is analysed by ICP (Inductively Coupled Plasma) coupled to an atomic emission spectrophotometry (AES) detector. During its introduction into the ICP-AES, the elements present in the solution will be excited on contact with the plasma. During their return to their ground state, they will emit radiation, the wavelength of which will be representative of their chemical nature. Thus, by detecting the intensity of the radiation at the wavelength corresponding to magnesium, it will be possible to determine its content in the solution.

Co Level by Spectrophotometry in Cobalt Acetylacetonate

Cobalt acetylacetonate is hydrolysed in an acidified aqueous solution (for example with nitric acid). Once the cobalt acetylacetonate has completely dissolved by stirring, the solution thus obtained is analysed by ICP (Inductively Coupled Plasma) coupled to an atomic emission spectrophotometry (AES) detector. During its introduction into the ICP-AES, the elements present in the solution will be excited on contact with the plasma. During their return to their ground state, they will emit radiation, the wavelength of which will be representative of their chemical nature. Thus, by detecting the intensity of the radiation at the wavelength corresponding to cobalt, it will be possible to determine its content in the solution.

### Example 1

Magnesium acetylacetonate dihydrate is synthesized.

The reaction is carried out starting from magnesium hydroxide and a molar excess of acetylacetone (acetylacetone/magnesium hydroxide molar ratio=6).

On conclusion of the reaction, the aqueous filtrate containing the excess acetylacetone and dissolved magnesium acetylacetonate is entirely recycled in a following synthesis. Thus, the operation is repeated 7 times.

The initial aqueous dilution is advantageously calculated in order to be able to retain a homogeneous aqueous phase for the filtrate. This limit is reached when the weight of acetylacetone in the water approaches 15%. When this limit is exceeded, a supernatant organic phase will appear in the filtrate. This situation can make the filtration more difficult. The experimental conditions are as follows:

Reaction 1:

The magnesium hydroxide is suspended in water in a 11 reactor and the acetylacetone is added in full with stirring to this aqueous suspension. The reaction time is 4 hours. Reaction very slightly exothermic (+10° C. the 1st half hour).

Raw water: 750 ml

+Magnesium hydroxide Mg(OH)₂: ˜19 g

(0.32 mol×58.32 g/mol×1/0.99 (purity)=18.85 g)

+Acetylacetone: ˜200 ml

(0.32 mol×6(acetylacetone/Mg)×100.12 g/mol×1/0.995 (purity)×1/0.975 (density)=198.15 ml)

Reaction 2:

The magnesium hydroxide is added in full to the filtrate of reaction 1 (first without stirring) and then the acetylacetone and the additional water (correction of the loss associated with the drying in so far as, in this example, there is no recycling of the condensate) are added with stirring to this suspension. The reaction time is 4 hours.

Filtrate reaction 1: ˜840 ml

Water: ˜40 ml (compensation for significant loss associated with the drying)

+Magnesium hydroxide Mg(OH)₂: ˜19 g

(0.32 mol×58.32 g/mol×1/0.99 (purity)=18.85 g)

+Acetylacetone: ˜70 ml (compensation for slight loss ˜4 ml associated with the drying)

(0.32 mol×2(acetylacetone/Mg)×100.12 g/mol×1/0.995 (purity)×1/0.975 (density)=66.05 ml)

Reaction 3:

The acetylacetone and the additional water are added to the filtrate from reaction 2 with stirring (the reverse, namely the filtrate to the acetylacetone and additional water, is also possible). The magnesium hydroxide is added in small portions to the water/acetylacetone reaction medium (slightly two-phase but well stirred at ˜500 rpm). The addition (by spatula) lasts between 10-15 min. The total reaction time (4 h) encompasses this addition time.

Filtrate reaction 2: ˜840 ml

Water: ˜40 ml

+Acetylacetone: ˜70 ml

+Magnesium hydroxide Mg(OH)₂: ˜19 g

Reaction 4:

Same protocol as reaction 3.

Filtrate reaction 3: ˜840 ml

Water: ˜40 ml

+Acetylacetone AAH: ˜70 ml

+Magnesium hydroxide Mg(OH)₂: ˜19 g

Reaction 5:

Same protocol as reaction 4.

Reaction 6:

Same protocol as reaction 5.

Reaction 7:

Same protocol as reaction 6.

Reaction 8:

Same protocol as reaction 7.

After each reaction, the filtration is carried out on a sintered glass (No. 3). The filtration of the white precipitate is quick and easy. The product is left for 15-20 minutes under a vacuum of approximately 150 mbar. The filtrate is clear and coloured golden-yellow with a slight odour of β-diketone. The pH of this filtrate, measured with pH paper, is approximately 6. The volume collected is ˜840 ml. No washing is carried out on the filter cake, the liquid filtrate being directly recovered for the following synthesis.

Drying

The magnesium acetylacetonate obtained is dried in order to obtain either an anhydrous form or a dihydrated form with, for the latter, less severe drying conditions.

Thus, for an anhydrous form, the conditions of the drying in an oven are a temperature of 50° C. under a vacuum of approximately 60 mbar regulated with slight nitrogen flushing, until a constant weight is obtained.

Thus, for a dihydrated form, the conditions of the drying in an oven are a temperature of 50° C. under a vacuum of approximately 500 mbar regulated with slight nitrogen flushing, until a constant weight is obtained.

The characterizations of the magnesium acetylacetonate obtained are given in Table 1.

TABLE 1

| | Regulated | % Mg | | | | |
|---|---|---|---|---|---|---|
| | drying with | ICP-AES | | | | |
| | slight nitrogen | (optical | | % H₂O | | |
| | flushing to | emission | | Karl- | Loss on stoving | % C |
| | constant weight | spectrometry) | Complexometry | Fischer | (2 h at 100° C.) | Microanalysis |
| Reaction 3 | 50° C.-60 mbar | 10.83 | 10.92 | 0.2 | | 53.4 |
| Reaction 7 | 50° C.-60 mbar | | 10.96 | | | |
| Anhydrous | | 10.92 | | 0.00 | | 53.98 |
| MgAA₂ | | | | | | |
| (theory) | | | | | | |
| Reaction 7 | 50° C.-500 mbar | | 9.44 | | 14.2 | |

| | | | |
|---|---|---|---|
| MgAA₂ | 9.40 | 13.94 | 46.46 |
| dihydrate | | | |
| (theory) | | | |

The yield of magnesium acetylacetonate (anhydrous or dihydrate) calculated from the amount of magnesium hydroxide introduced is given for each reaction in Table 2. For example, the yield for reaction 7 is 96% (79.5 g of product obtained) if it is considered that the product is in the dihydrate form (258.55 g/mol). The theoretical level of magnesium is 9.40%. This yield illustrates the loss of magnesium acetylacetonate by dissolution in the filtrates and the advantage of recovering these filtrates.

TABLE 2

| | Yield (% by |
|---|---|
| Reaction | weight) |
| 1 | 86 |
| 2 | 82 |
| 3 | 92 |
| 4 | 92 |
| 5 | 94 |
| 6 | 95 |
| 7 | 96 |
| 8 | 94 |

### Example 2

Cobalt acetylacetonate dihydrate is synthesized.

The reaction is carried out starting from cobalt(II) hydroxide and a molar excess of acetylacetone (acetylacetone/cobalt hydroxide molar ratio=6).

On conclusion of the first reaction, the aqueous filtrate containing the excess acetylacetone and dissolved cobalt acetylacetonate is entirely recycled in a following synthesis. Thus, the operation is repeated 3 times.

The initial aqueous dilution is advantageously calculated in order to be able to retain a homogeneous aqueous phase for the filtrate. This limit is reached when the weight of acetylacetone in the water approaches 15%. When this limit is exceeded, a supernatant organic phase will appear in the filtrate. This situation can make the filtration more difficult. The experimental conditions are as follows:

Reaction 1:

The cobalt hydroxide is suspended in water in a 11 reactor and the acetylacetone is added in full with stirring to this aqueous suspension. The reaction time is 4 hours. Slightly exothermic reaction.

Raw water: 750 ml

+Cobalt hydroxide Co(OH)₂: ˜30 g

(0.32 mol×92.95 g/mol×1/0.99 (purity)=30.04 g)

+Acetylacetone: ˜200 ml

(0.32 mol×6(acetylacetone/Co)×100.12 g/mol×1/0.995 (purity)×1/0.975 (density)=198.15 ml)

Reaction 2:

The Co(II) hydroxide is added in full to the filtrate of reaction 1 (suspended with stirring) and then the acetylacetone and the additional water (correction of the loss associated with the drying in so far as, in this example, there is no recycling of the condensate) are added with stirring to this suspension. The reaction time is 4 hours at ambient temperature with stirring (˜500 rpm).

Filtrate reaction 1: ˜840 ml

Water: ˜40 ml (compensation for significant loss associated with the drying)

+Cobalt hydroxide Co(OH)₂: ˜30 g

(0.32 mol×92.95 g/mol×1/0.99 (purity)=30.04 g)

+Acetylacetone: ˜70 ml (compensation for slight loss ˜4 ml associated with the drying in so far as, in this example, there is no recycling of the condensate)

(0.32 mol×2(acetylacetone/Co)×100.12 g/mol×1/0.995 (purity)×1/0.975 (density)=66.05 ml)

Reaction 3:

Same protocol as reaction 2.

Filtrate reaction 2: ˜840 ml

Water: ˜40 ml

+Cobalt hydroxide Co(OH)₂: ˜30 g

+Acetylacetone AAH: ˜70 ml

After each reaction, the filtration is carried out on a sintered glass (No. 3 or 4). The filtration of the salmon pink precipitate is quick and easy. The product is left for 15-20 minutes under bench vacuum. The filtrate is clear and coloured red with a slight odour of β-diketone. The pH of this filtrate is approximately 5 (pH paper). The volume collected is ˜840 ml. No washing and recovery of the filtrate for the following synthesis.

Drying

The cobalt acetylacetonate obtained is dried in order to obtain an anhydrous form. The conditions of drying in an oven are a temperature of 50° C. under a vacuum of approximately 250 mbar regulated with slight air or nitrogen flushing, to a constant weight.

The colour of the product obtained (salmon pink) makes it possible to observe that the product obtained is indeed cobalt acetylacetonate dihydrate.

The characterizations of the cobalt acetylacetonate obtained are given in Table 3.

TABLE 3

| | Regulated | % Co | | | |
|---|---|---|---|---|---|
| | drying with | ICP-AES | | | |
| | slight nitrogen | (optical | | % H₂O | Appearance |
| | flushing to | emission | | Loss on stoving | Colour of |
| | constant weight | spectrometry) | Complexometry | (4 h at 75-80° C.) | the crystals |
| Reaction 2 | 50° C.-250 mbar | 20.16 | 20.08 | 12.0 | salmon pink |

| | | | |
|---|---|---|---|
| CoAA₂ | 20.10 | 12.29 | salmon pink |
| dihydrate | | | |
| (theory) | | | |
| Anhydrous | 22.92 | 0.00 | burgundy |
| CoAA₂ | | | purple |
| (theory) | | | |

The yield of cobalt(II) acetylacetonate calculated from the amount of cobalt(II) hydroxide introduced is given for each reaction in Table 4. For example, the yield for reaction 3 is 97% (91 g of product obtained) if it is considered that the product is in the dihydrated form (293.18 g/mol). The theoretical level of cobalt is 20.10%.

TABLE 4

| | Yield (% by |
|---|---|
| Reaction | weight) |
| 1 | 94 |
| 2 | 98 |
| 3 | 97 |

1.-15. (canceled)

16. A process for the preparation of an acetylacetonate of a chemical element Me, the acetylacetonate being hydrated, anhydrous or both hydrated and anhydrous, where the chemical element Me is selected from the group consisting of alkaline earth metals, transition metals and lanthanides, comprising:
reacting Me oxide or hydroxide in a solid form with acetylacetone, in an aqueous medium, the acetylacetone being in excess with respect to the Me oxide or hydroxide.

17. The process according to claim 16, wherein an acetylacetone/Me oxide or hydroxide molar ratio is greater than 2 when the Me oxide or hydroxide is divalent or greater than 3 when the Me oxide or hydroxide is trivalent.

18. The process according to claim 16, wherein the chemical element Me is selected from alkaline earth metals selected from the group consisting of magnesium, calcium, strontium and barium.

19. The process according to claim 16, wherein the chemical element Me is selected from transition metals selected from the group consisting of cobalt, nickel, copper and zinc.

20. The process according to claim 16, wherein the chemical element Me is selected from lanthanides selected from the group consisting of lanthanum, cerium, praseodymium and neodymium.

21. The process according to claim 16, wherein Me is selected from the group consisting of cobalt, magnesium, nickel, calcium, neodymium and zinc.

22. The process according to claim 19, wherein Me is cobalt and cobalt acetylacetonate dihydrate is obtained by reacting cobalt(II) hydroxide and acetylacetone in water.

23. The process according to claim 18, wherein Me is magnesium and magnesium acetylacetonate dihydrate is obtained by reacting magnesium(II) hydroxide or magnesium oxide and acetylacetone in water.

24. A continuous batchwise process for a synthesis of hydrated or anhydrous acetylacetonate of a chemical element Me, where the chemical element Me is selected from the group consisting of alkaline earth metals, transition metals and lanthanides, comprising:
n steps of (i) reacting an Me oxide or hydroxide in a solid form with acetylacetone, in an aqueous medium, the acetylacetone being in excess with respect to the Me oxide or hydroxide;
n steps of (i′) filtering the reaction mixture after each step (i); and
n−1 steps of recovering a liquid filtrate comprising water, the excess acetylacetone and a fraction of dissolved Me acetylacetonate, and recycling the liquid filtrate to a next step (i),
wherein n is greater than or equal to 2.

25. The continuous batchwise process according to claim 24, wherein, for each step (i), an acetylacetone/Me oxide or hydroxide molar ratio is greater than 2 when the Me oxide or hydroxide is divalent and greater than 3 when the Me oxide or hydroxide is trivalent.

26. The continuous batchwise process according to claim 24, wherein a weight of acetylacetone in an aqueous phase of the liquid filtrate is less than or equal to 15% of a weight of the aqueous phase.

27. The continuous batchwise process according to claim 24, wherein the chemical element Me is selected from the group consisting of cobalt, magnesium, nickel, calcium, neodymium and zinc.

28. The continuous batchwise process according to claim 24, wherein hydrated acetylacetonate of the chemical element Me recovered after each filtration step (i′) is dried.

29. A process for a preparation of hydrated or anhydrous acetylacetonate of a chemical element Me, where the chemical element Me is selected from the group consisting of alkaline earth metals, transition metals and lanthanides, the process comprising:
reacting an Me oxide or hydroxide and acetylacetone,
wherein Me acetylacetonate, a liquid filtrate containing acetylacetone in an aqueous phase, and a condensate are obtained, the liquid filtrate being capable of being used for a new preparation of acetylacetonate of the element Me.

30. The process according to claim 29, wherein the reacting step comprises a first stage of reacting the Me oxide or hydroxide in a solid form with acetylacetone, in an aqueous medium, the acetylacetone being in excess with respect to the Me oxide or hydroxide, and a second stage of filtering.
